(19) United States
(12) Patent Application Publication
(10) Pub. No.: US 2021/0102891 A1
(43) Pub. Date: Apr. 8, 2021

(54) METHOD AND APPARATUS FOR DETERMINING WATER CONTENT IN A HYDROCARBON FLUID

(71) Applicant: Parker Hannifin EMEA S.à.r.l., Etoy (CH)

(72) Inventor: Anthony P. Foord, Worthing (GB)

(73) Assignee: Parker Hannifin EMEA S.à.r.l., Etoy (CH)

(21) Appl. No.: 17/106,229

(22) Filed: Nov. 30, 2020

Related U.S. Application Data
(63) Continuation of application No. PCT/EP2019/056885, filed on Mar. 19, 2019.

(30) Foreign Application Priority Data
Jul. 4, 2018 (GB) 1810976.9

(51) Int. Cl. G01N 21/3577; G01N 21/3554; G01N 33/28; G01N 21/552

(52) U.S. Cl. CPC G01N 21/3577; G01N 21/552; G01N 33/2847; G01N 21/3554

(57) ABSTRACT

A method and apparatus for measuring water content in a hydrocarbon fluid is provided. The method comprises: taking a first infrared measurement of the hydrocarbon fluid at a first temperature across at least one infrared absorption wavelength of water; taking a second infrared measurement of the hydrocarbon fluid at a second temperature across the at least one infrared absorption wavelength of water, the second temperature being different to the first temperature; determining a change between said first and second infrared measurements; and using the change between said first and second infrared measurements to determine the water content in the hydrocarbon fluid. Apparatus configurations for performing the method are described.

### FIELD OF INVENTION

The present invention relates to methods and apparatus for measuring water content in hydrocarbon fluids such as lubricants, fuels, and oils.

### BACKGROUND

Chemical titration methods for determining water content in hydrocarbon fluids are known in the art. “Karl Fischer” titration is a classic titration method in analytical chemistry that uses coulometric or volumetric titration to determine trace amounts of water in a sample. Another example of a prior art chemical (calcium hydride) based method for determining water in hydrocarbon fluids is “Digicell” sold by Parker-Hannifin Corporation of Cleveland, Ohio.

One problem with such chemical-based methods, from a supplier's perspective, is that they require the shipping of consumables to a user for each test. From a customer's perspective, such consumables must be purchased and may cause delay while waiting for a new supply of consumables. Another problem is that samples need to be extracted for testing to avoid contamination of the hydrocarbon fluid. As such, it is desired to provide a method of determining water in hydrocarbon fluids which does not require chemical additives, and which may also be used for inline testing without contamination of the hydrocarbon fluid.

One alternative to the use of chemical-based testing is to use infrared spectroscopy. Infrared spectroscopy allows a quick and convenient measurement of dissolved and emulsified water in lubricants, fuels and hydraulic oils compared to chemical methods like Karl Fischer titration. However, prior art infrared methods can have relatively poor accuracy, particularly at low water content levels required for many applications. One reason for the relatively poor accuracy is because of offsets due to the relatively strong infrared absorption of hydrocarbons at the water absorption wavelength and, in the case of engine lubricants, very strong infrared absorption from soot across all wavelengths.

Using infrared absorption measurement to accurately determine the water content of oils and fuels is thus problematic because the absorption due to water is relatively weak and overlaps with a strong hydrocarbon infrared absorption and (in the case of engine lubricants) strong broadband absorption from soot. Past approaches rely on: (a) subtracting the hydrocarbon absorption measured for a dry reference sample of the same oil (requiring knowledge of the specific oil being tested and prior calibration for this); (b) extrapolation of the infrared absorption due to soot from other wavelengths requiring measurements to be made at one or more other wavelengths and offering limited accuracy; or (c) accepting that infrared measurements of water are qualitative rather than quantitative.

It should also be noted that for present infrared methods where a reference measurement made on a new sample is subtracted, new samples commonly contain an unknown and variable amount of water (depending on how they have been stored, etc.). This introduces an error into infrared measurements unless it is corrected for by determining the water content of the new sample by some other method, in which case the infrared measurement is no longer independent but is reliant on the accuracy of the other method.

A publication entitled “Beyond Karl Fischer: Titration Methods” (LabManager.com, 2017) describes two alternatives to Karl Fischer titration: one relying on infrared measurements and one using a drying method. The infrared method is described as requiring an extensive library of oil-specific calibrations.

Parker-Hannifin Corporation offers several infrared-based products for measuring water in hydrocarbon fluids. “WaterSCAN” is a current infrared-based product for measuring water in oil. “FTIR” is another current infrared-based product for measuring water in oil. “ATR” is a new infrared-based product for measuring water in oil. These three products are able to make infrared determinations of water content in the presence of soot, but all rely on more or less sophisticated extrapolations of the infrared absorption due to soot from measurements at other wavelengths.

Furthermore, the infrared properties of soot depend on fuel type and engine conditions (and therefore engine type). Accordingly, extrapolating soot absorption measured at one wavelength as a correction to another wavelength can only be approximate.

It is desired to provide an apparatus and method which does not require a separate calibration for every specific hydrocarbon fluid. Furthermore, it is desired to provide an apparatus and method which is capable of more accurate measurement of water content at low water content and/or in the presence of soot.

An aim of the present invention is to solve the problems outlined above with regard to the prior art methods and systems.

### SUMMARY OF INVENTION

An unusual feature of the infrared absorption of water is its very large temperature coefficient, whereas absorption due to hydrocarbons and soot is essentially unaffected by temperature. It has also been noted that the infrared absorptions of standard oil additives have no significant temperature coefficient. The present invention utilizes this difference to provide a means for precisely determining the part of the infrared absorption due to water by making measurements at two or more temperatures. It is applicable to laboratory type instruments and online sensors.

The concept of the invention is thus to determine the water content of a hydrocarbon fluid, such as such as a lubricant, fuel, or oil, by measuring the infrared absorption due to water at two or more temperatures. The infrared absorption of water has a large temperature coefficient (both as free water and when dissolved in oil) whereas the overlapping infrared absorption due to hydrocarbons and soot) is substantially independent of temperature. The change in infrared absorption over temperature at the water wavelength is therefore due almost solely to the water content and this allows the water content to be accurately determined.

According to a first aspect of the invention, there is provided a method of measuring water content in a hydrocarbon fluid, the method comprising:
a. taking a first infrared measurement of the hydrocarbon fluid at a first temperature across at least one infrared absorption wavelength of water;
b. taking a second infrared measurement of the hydrocarbon fluid at a second temperature across the at least one infrared absorption wavelength of water, the second temperature being different to the first temperature;
c. determining a change between said first and second infrared measurements; and
d. using the change between said first and second infrared measurements to determine the water content in the hydrocarbon fluid.
   One possible infrared absorption wavelength which can be used for the infrared absorption wavelength of water in the methodology is the broad region around 3400 cm⁻¹ (2.94 micrometres). The first and second infrared measurements may each comprise taking an infrared spectrum of the hydrocarbon fluid over a wavelength range which includes at least one infrared absorption wavelength of water. Alternatively, the infrared measurements may be non-dispersive infrared measurements rather than spectrometer measurements. Furthermore, the first and second infrared measurements may be transmittance type measurements through a sample of the hydrocarbon fluid. Alternatively, the first and second infrared measurements may be attenuated total reflectance (ATR) infrared measurements.

The change between first and second infrared measurements may be calculated by simple subtraction of the two infrared measurements. Alternatively, infrared measurements may be taken over more than two temperatures or a gradually changing temperature. The change may then be calculated using a gradient of changing infrared intensity with temperature. As such, it will be evident that variants are envisaged for the type of infrared measurements which can be used and the way in which the change in measurement results with temperature can be used to determine water content.

The method may be used to determine water content in a range of different hydrocarbon fluids including lubricants, fuels, and oils. The method is particularly useful for use with hydrocarbon fluids which comprise soot (as a contaminant) as these hydrocarbon fluids are particularly problematic for determination of water content using prior art methods.

Calibration data can be utilized to determine the water content in the hydrocarbon fluid from the first and second infrared spectroscopy measurements, the calibration data generated from samples of hydrocarbon fluid having known concentrations of water. In this case, for each family of hydrocarbon fluids, the same calibration data may be utilized rather than requiring calibration data for each individual type of hydrocarbon fluid. As such, huge libraries of calibration data for each individual hydrocarbon fluid are not required. The methodology using infrared measurements at different temperatures is less sensitive to changes in the precise formulation of hydrocarbon fluid. While differences may occur for very different hydrocarbon fluid samples, such differences can be dealt with using a much smaller library of calibration data for families of fluid types rather than individual samples.

One calibration or correction factor which may be useful to apply in the present methodology is to compensation for the expansion or contraction of a hydrocarbon fluid with varying temperature, which can affect the path length of infrared light or the density of the hydrocarbon fluid. A correction can thus be applied to account for expansion or contraction of the hydrocarbon fluid between the first and second measurement temperatures. For example, a correction can be applied by taking an infrared measurement at the first and second temperatures at a wavelength associated with the hydrocarbon fluid and subtracting a change in the infrared measurement to correct for changes in absorption due to expansion or contraction of the hydrocarbon fluid. Alternatively, the mechanical configuration of the apparatus can be designed to ensure that temperature induced expansion does not lead to errors in the infrared measurements.

According to another aspect of the present invention there is provided an infrared apparatus configured to perform the method as previously described. Such an infrared apparatus comprises:
a. an infrared device configured to take infrared measurements of the hydrocarbon fluid;
b. a heater configured to heat and/or cool the hydrocarbon fluid;
c. a controller configured to take a first infrared measurement at a first temperature across at least one infrared absorption wavelength of water and to take a second infrared measurement at a second temperature across the at least one infrared absorption wavelength of water, the second temperature being different to the first temperature; and
d. a processing unit configured to determining a change between said first and second infrared measurements and use the change between said first and second infrared measurements to determine the water content in the hydrocarbon fluid.

It is also envisaged that standard infrared equipment could be utilized to take the infrared measurements at different temperatures and a computer program can be provided to perform the data analysis required to implement the method as described herein. As such, according to yet another aspect of the present invention there is provided a computer readable storage medium comprising computer-executable instructions which, when executed, configure one or more processors to perform the method as described herein. As an alternative to a computer readable storage medium, the computer program can be provided for download via the internet.

### BRIEF DESCRIPTION OF THE DRAWINGS

Embodiments of the present invention are described by way of example only with reference to the accompanying drawings in which:

FIG. 1 shows a schematic of an infrared apparatus for measuring water content in a hydrocarbon fluid as described herein;

FIG. 2 shows another schematic of an infrared apparatus for measuring water content in a hydrocarbon fluid as described herein;

FIG. 3 shows infrared spectra (absorption vs wavenumber) for a sample of marine lubricating oil containing water measured at two different temperatures;

FIG. 4 shows a section of the infrared spectra of FIG. 3 between 3000 and 4000 cm⁻¹, the region at which infrared absorption attributable to water content occurs;

FIG. 5 shows the difference between the two spectra taken at two different temperatures illustrating a large difference between 3000 and 4000 cm⁻¹ due to infrared absorption by water;

FIG. 6 shows the normalized infrared absorption at the water wavelength vs water content (ppm) measured at two different temperatures (40° C. and 70° C.); and

FIG. 7 shows the difference in absorbance vs water content (ppm) for the two different temperature measurements of FIG. 6.

### DETAILED DESCRIPTION

As described in the Summary of Invention section above, a method and apparatus is provided to determine the water content of a hydrocarbon fluid, such as a lubricant, fuel, or oil, by measuring infrared absorption due to water at two or more temperatures and using a change in the infrared measurements with temperature to determine water content.

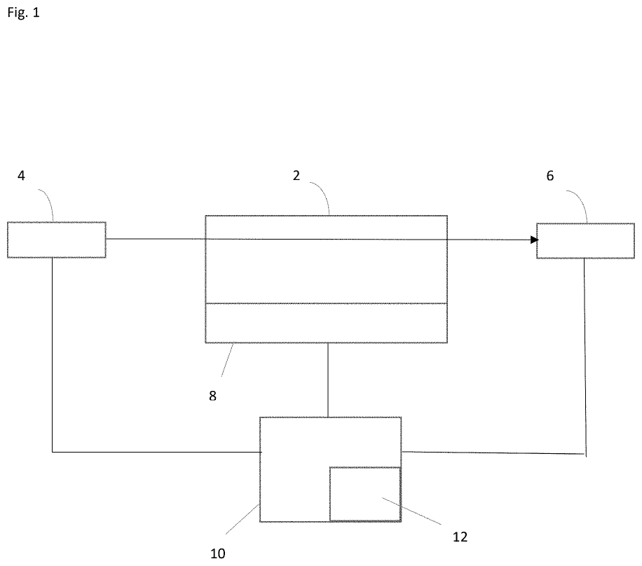

FIG. 1 shows a schematic of the basic components of an infrared apparatus for measuring water content in a hydrocarbon fluid. The apparatus comprises: a hydrocarbon fluid cell 2; an infrared device comprising an infrared emitter 4 and am infrared detector 6; a heater 8 configured to heat and/or cool the hydrocarbon fluid; a controller 10 configured to take a first infrared measurement at a first temperature across at least one infrared absorption wavelength of water and to take a second infrared measurement at a second temperature across the at least one infrared absorption wavelength of water, the second temperature being different to the first temperature; and a processing unit 12 configured to determining a change between said first and second infrared measurements and use the change between said first and second infrared measurements to determine the water content in the hydrocarbon fluid. Such an apparatus can be provided as a handheld, desktop, or inline system.

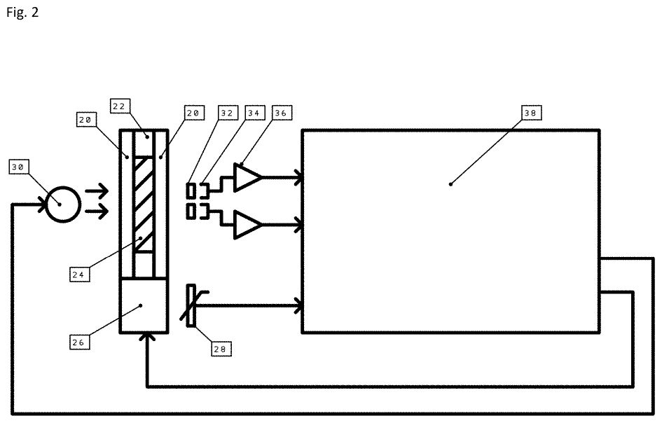

FIG. 2 shows another schematic of an infrared apparatus for measuring water content in a hydrocarbon fluid as described herein. The illustrated example is an NDIR (non-dispersive infrared) type instrument with a filter for each channel rather than a spectrometer. A two-channel system is shown including a water channel and a reference channel (optional) used to compensate for thermal expansion when taking measurements at different temperatures. Different infrared filters are used to extract water and reference signals for the two infrared channels. The basic components of the apparatus include: a sample holder including IR windows 20 and spacers 22 defining a space or channel for a sample 24; a heater 26 and a temperature sensor 28; an infrared device including an IR source 30, IR filters 32, IR detectors 34, and amplifiers 36; and a control and display unit 38 for controlling the temperature of the sample, taking IR measurements at different sample temperatures, and calculating and displaying results.

The apparatus may be configured to take offline sample measurements, e.g. hand held or desk top devices, or integrated into a system to take online measurements. Applications include engine systems and bunkered fuel oil.

Heating of hydrocarbon samples may be via electrical heating, thermoelectric heating, or Peltier heating. The latter provides the option to cool the hydrocarbon fluid as well as heating. Furthermore, if a system temperature varies naturally during operation then the natural integral variation of a system temperature can be used to capture infrared measurements at two different times.

The basic methodology for acquiring and processing infrared measurements is described below.

The light transmitted through a sample drops off exponentially with distance, or with increasing concentration of the absorbing species (“Beer-Lambert Law”). So, for example, if a given concentration transmits 10% of the incident light then twice that concentration would transmit 1% (10% of 10%) and three times the concentration would transmit 0.1% (10% of 10% of 10%).

It is convenient and common practice in spectroscopy to linearize this behaviour by taking the log (to base 10) of the ratio of the incident intensity (I₀) to the transmitted intensity (Iₜ) and working in “absorbance units” (AU), i.e.

A=log(I₀/Iₜ)

So, 10% transmission would give 1.0 AU (log[1/0.1]) while 1% transmission would be 2.0 AU (log[1/0.01]) and so on. Working in AU ratios of absorbance become subtractions, and that is how the calculations have been set out below. It is worth bearing in mind that taking logs at this point isn't fundamental to the calculation, just convenient and conventional. It would be equally possible to divide the transmission values and perform the log conversion on the final value.

### Basic Calculation (No Compensation for Expansion)

Water absorbs infrared more strongly (higher AU) at lower temperatures. Taking the simplest case of measurement at just two temperatures and calling the absorbance due to water at the hotter temp A_{WH} and the absorbance at the colder temp A_{WC} then the water content is just:

Water content=(A_{WC}−A_{WH})/A_{CAL}

A_{CAL} is a calibration factor determined previously by making the same measurement (i.e. same two temperatures) on a sample containing a known quantity of water.

A_{CAL}=(A_{WC}−A_{WH})/water content

The calculation is simple and is similar to that for an instrument measuring at a single temperature and relying on subtracting a previously determined baseline that is stored in a library:

Water content=(A_{W}−A_{WLIB})/A_{CAL}

The problem with the conventional method is that it requires the hydrocarbon fluid formulation to be known and to have previously made and stored a baseline measurement for that particular formulation. Furthermore, in practice using the conventional approach leads to errors due to the fact that a comparison is being made between a used, sooty hydrocarbon fluid and a new, clean hydrocarbon fluid typically used in the calibration. As also previously stated, it should also be noted that for prior art infrared methods where a reference measurement made on a new sample is subtracted, new samples commonly contain an unknown and variable amount of water (depending on how they have been stored, etc.). This introduces an error into infrared measurements unless it is corrected for by determining the water content of the new sample by some other method, in which case the infrared measurement is no longer independent but is reliant on the accuracy of the other method. In contrast, in the two (or more) temperature method the sample acts as its own baseline.

Calculation with Compensation for Expansion

Oils expand and become less dense when heated and so if the transmission path length is fixed the infrared light will actually be passing through smaller amount of oil at higher temperatures, reducing the baseline absorption. The measurement cell may also expand, increasing the path length and the absorption. This will tend to reduce the effect of the oil expansion, but only partially since typical cell materials expand much less than oil. The overall change in effective path length affects all wavelengths equally and so it is simple enough to measure the effect at some other reference wavelength (using the same hot and cold temps) and use this to compensate the water measurement:

A_{CORR}=A_{REFC}−A_{REFH}

and then:

Water content=(A_{WC}−A_{WH}−A_{CORR})/A_{CAL}

The correction needs to be applied consistently, i.e. if it is applied to the measurement it should also have been applied to the initial determination of A_{CAL}.

### Temperature Compensated Cell

Compensation for expansion based on a second measurement wavelength as above is one approach. However, an alternative method is to design a measurement cell such that the cell expansion compensates for the oil expansion. One advantage of this approach is that it removes the need for a second channel in a very basic instrument that relies on filters to select measurement wavelengths.

Thermal expansion coefficients for liquids are specified as volumetric coefficients and mineral oils and fuels are typically in the range 7×10⁻⁴/K to 1×10⁻³/K. The fluid is free to expand (and reduce in density) and so the value we want to match with the linear expansion of the cell is approximately ⅓ of the volumetric coefficient, so 2.3×10⁻⁴/K to 3.3×10⁻⁴/K. Metals typically have linear expansion coefficients of around 20×10⁻⁶/K (˜ 1/10 the desired value) and PTFE (which is commonly used for spacing IR transmission cells) has a coefficient of around 120×10⁻⁶/K (˜½ the desired value). Sapphire (a possible material for the IR window) has a much lower coefficient of around 5×10⁻⁶/K.

A solution to match the lower expansion coefficients of solids to those of oils is to make the cell spacer proportionally thicker. For example, if we wanted a 0.2 mm measurement path though oil (a good value for marine lubricant measurements) we could roughly offset the decrease in density of the oil with the expansion of a 2 mm to 2.5 mm metal spacer. This, of course, gives far too great a path length through the oil but this can be taken up by a second, low expansion coefficient mechanical part that is mounted so that it expands in the opposite direction to the metal spacer. One way of realising this would be to mount a (very roughly) 1.8 mm thick sapphire window by its outer face. Or, alternatively, a piece of sapphire of this about this thickness could be inserted into the cell. This construction is compatible with the cell design in patent application GB2545541A “Sample Testing Method and Apparatus”.

While the aforementioned constructions provide some examples of temperature compensated cell constructions, it should be noted that other constructions are possible. The main point is that that there is a density change in oil with temperature and that one way of dealing with this is a compensating expansion of the path length.

### Examples

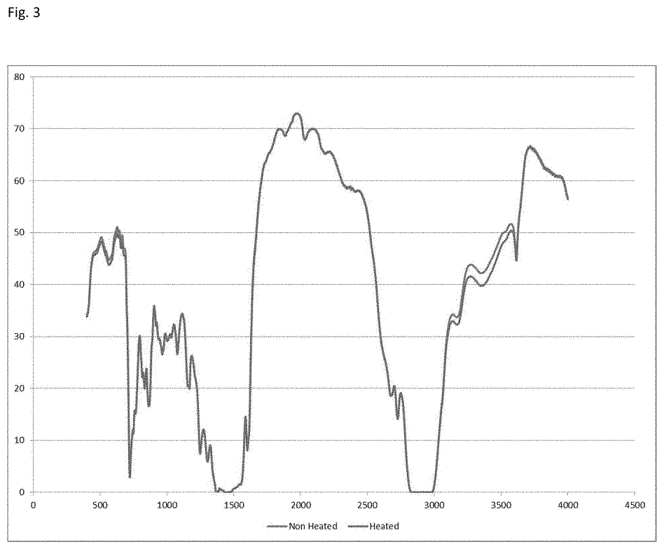

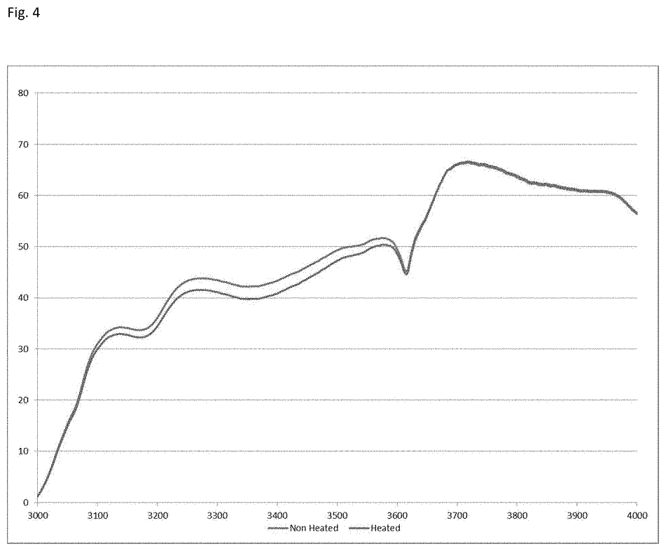

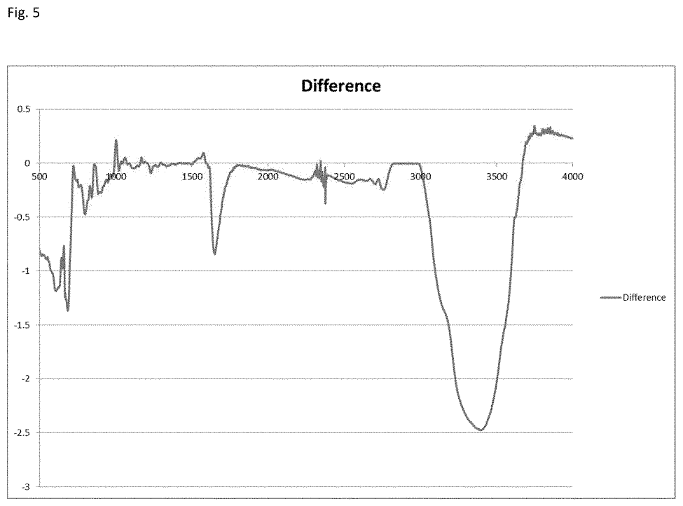

FIG. 3 shows infrared spectra (absorption vs wavenumber) for a sample of marine lubricating oil containing water measured at two different temperatures. The spectra overlap except in regions associated with water absorption. As previously described, while infrared absorption due to hydrocarbon fluids is relatively insensitive to changes in temperature, infrared absorption due to water changes significantly with temperature. In this regard, FIG. 4 shows a section of the infrared spectra of FIG. 3 between 3000 and 4000 cm⁻¹, a region at which infrared absorption is attributable to water content. FIG. 5 shows the difference between the two spectra taken at two different temperatures illustrating a large difference between 3000 and 4000 cm⁻¹ due to infrared absorption by water.

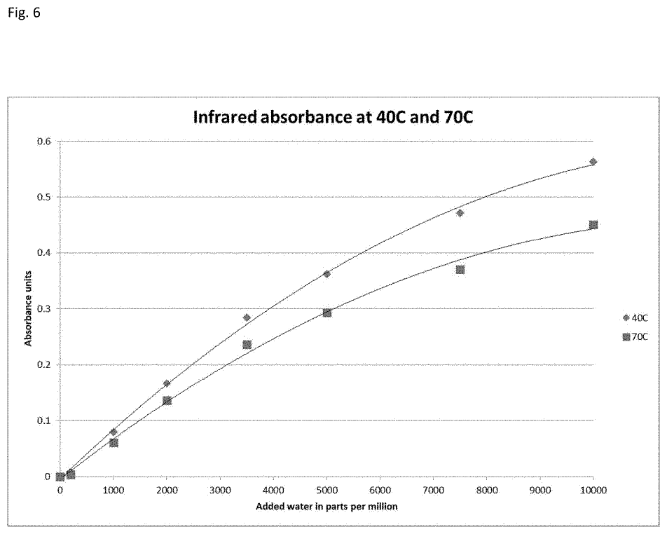

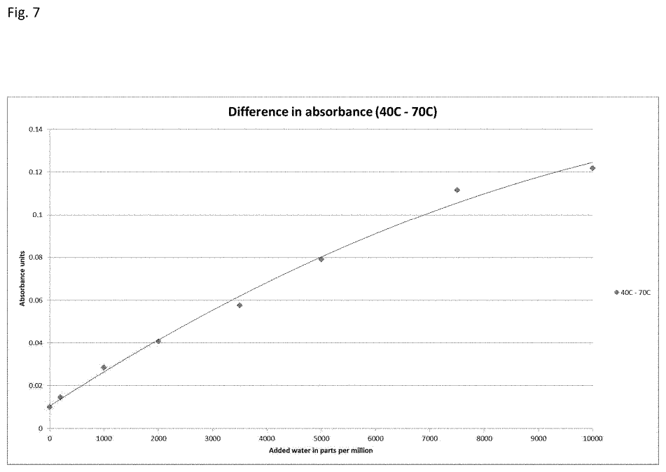

FIG. 6 shows the normalized infrared absorption at a water wavelength vs water content (ppm) measured at two different temperatures (40° C. and 70° C.). FIG. 7 shows the difference in absorbance vs water content (ppm) for the two different temperature measurements of FIG. 6. In the temperature difference data, the graph remains relatively smooth and linear up to about 7500 ppm. This substantially linear response over a wide water content range is advantageous for use of the methodology in a wide range of applications and a wide range of hydrocarbon fluids have very different water contents. For example, for certain applications the upper limit for water content is 0.25%. Jet fuel is limited to no more than 50 ppm free water. In contrast, marine lubricant includes many additives and has a much higher water content than standard hydrocarbon fluids. Additives can change according to specific formulations. The present invention enables water content to be determined for a range of different formulations without requiring new calibrations for each formulation. While the data in FIG. 7 shows a substantially linear response, it should be noted that this isn't a strict requirement and a monotonic increase with temperature would suffice.

It will be appreciated that while the invention has been described in relation to certain embodiments, it will be appreciated that various alternative embodiments can be provided without departing from the scope of the invention which is defined by the appending claims.

1. A method of measuring water content in a hydrocarbon fluid, the method comprising:
i. taking a first infrared measurement of the hydrocarbon fluid at a first temperature across at least one infrared absorption wavelength of water;
ii. taking a second infrared measurement of the hydrocarbon fluid at a second temperature across the at least one infrared absorption wavelength of water, the second temperature being different to the first temperature;
iii. determining a change between said first and second infrared measurements; and
iv. using the change between said first and second infrared measurements to determine the water content in the hydrocarbon fluid.

2. The method according to claim 1, wherein the first and second infrared measurements each comprise taking an infrared spectrum of the hydrocarbon fluid over a wavelength range which includes the at least one infrared absorption wavelength of water.

3. The method according to claim 2, wherein the first and second infrared measurements include measuring transmittance through a sample of the hydrocarbon fluid.

4. The method according to claim 1, wherein the first and second infrared measurements include measuring transmittance through a sample of the hydrocarbon fluid.

5. The method according to claim 2, wherein the first and second infrared measurements include measuring transmittance through a sample of the hydrocarbon fluid.

6. The method according to claim 1, wherein the first and second infrared measurements both include an attenuated total reflectance (ATR) infrared measurement.

7. The method according to claim 2, wherein the first and second infrared measurements both include an attenuated total reflectance (ATR) infrared measurement.

8. The method according to claim 1, wherein infrared measurements are taken over more than two temperatures or a gradually changing temperature.

9. The method according to claim 1, wherein the change is calculated by subtraction of two infrared measurements or using a gradient of changing infrared intensity with temperature.

10. The method according to claim 1, wherein the hydrocarbon fluid is one of a lubricant, a fuel, or an oil.

11. The method according to claim 1, wherein the hydrocarbon fluid comprises soot.

12. The method according to claim 1, wherein calibration data is utilized to determine the water content in the hydrocarbon fluid from the first and second infrared spectroscopy measurements, the calibration data generated from samples of hydrocarbon fluid having known concentrations of water.

13. The method according to claim 12, wherein for each family of hydrocarbon fluids, the same calibration data is used.

14. The method according to claim 1, wherein a correction is applied to account for expansion or contraction of the hydrocarbon fluid between the first and second temperatures.

15. The method according to claim 14, wherein the correction comprises taking an infrared measurement at the first and second temperatures at a wavelength associated with the hydrocarbon fluid and subtracting a change in the infrared measurement to correct for changes in absorption due to expansion or contraction of the hydrocarbon fluid.

16. An infrared apparatus for measuring water content in a hydrocarbon fluid, the infrared apparatus comprising:
i. an infrared device configured to take infrared measurements of the hydrocarbon fluid;
ii. a heater configured to heat and/or cool the hydrocarbon fluid;
iii. a controller configured to take a first infrared measurement at a first temperature across at least one infrared absorption wavelength of water and to take a second infrared measurement at a second temperature across the at least one infrared absorption wavelength of water, the second temperature being different to the first temperature; and
iv. a processing unit configured to determining a change between said first and second infrared measurements and use the change between said first and second infrared measurements to determine the water content in the hydrocarbon fluid.

17. The infrared apparatus according to claim 16, configured to perform the method of claim 1.

18. A computer readable storage medium comprising computer-executable instructions which, when executed, configure one or more processors to perform the method of claim 1 using data from the first and second infrared measurements to determine the water content in the hydrocarbon fluid.

19. A computer program comprising computer-executable instructions which, when executed, configure one or more processors to perform the method of claim 1 using data from the first and second infrared measurements to determine the water content in the hydrocarbon fluid.
